(19) United States
(12) Patent Application Publication
(10) Pub. No.: US 2026/0191514 A1
(43) Pub. Date: Jul. 9, 2026

(54) ARTICULATING IMAGING AND SAMPLE COLLECTION UNIT

(71) Applicant: Devicor Medical Products, Inc., Cincinnati, OH (US)

(72) Inventors: Maria B. ROMA, Cincinnati, OH (US); Lyle JAMES, Kettering, OH (US)

(21) Appl. No.: 19/556,201

(22) Filed: Mar. 4, 2026

Related U.S. Application Data
(63) Continuation of application No. PCT/US2024/048129, filed on Sep. 24, 2024.
(60) Provisional application No. 63/540,966, filed on Sep. 28, 2023.

(51) Int. Cl. A61B 10/00; A61B 10/02; A61B 90/50; G01N 23/04; G01N 23/083

(52) U.S. Cl. CPC A61B 10/0096; A61B 10/0275; A61B 90/50; G01N 23/04; G01N 23/083; G01N 2223/6126

(57) ABSTRACT

A biopsy system includes a biopsy device, a tissue transport tube, and a tissue sample holder. The biopsy device includes a probe, a needle, and a cutter. The needle extends distally from the probe. The cutter is movable relative to the needle to sever one or more tissue samples. The tissue transport tube is configured to couple to a portion of the biopsy device to communicate the one or more severed tissue samples from the biopsy device. The tissue sample holder is separate from the biopsy device and is configured to couple to the tissue transport tube to receive the one or more severed tissue samples. The tissue sample holder incudes a sample tray and an analysis assembly. The sample tray is configured to receive the one or more severed tissue samples. The sample tray has a fixed spatial relationship with respect to the analysis assembly.

### PRIORITY

This application claims priority to U.S. Provisional Application No. 63/540,966, entitled “Articulating Imaging and Sample Collection Unity,” filed on Sep. 28, 2023, the disclosure of which is hereby incorporated by reference herein.

### BACKGROUND

Biopsy samples have been obtained in a variety of ways in various medical procedures using a variety of devices. For instance, some biopsy devices may be fully operable by a user using a single hand, and with a single insertion, to capture one or more biopsy samples from a patient. In addition, some biopsy devices may be tethered to a vacuum module and/or control module, such as for communication of fluids (e.g., pressurized air, saline, atmospheric air, vacuum, etc.), for communication of power, and/or for communication of commands and the like. Other biopsy devices may be fully or at least partially operable without being tethered or otherwise connected with another device. Biopsy devices may be used under stereotactic guidance, ultrasound guidance, MRI guidance, PEM guidance, BSGI guidance, or otherwise.

Merely exemplary biopsy devices and biopsy system components are disclosed in U.S. Pat. No. 5,526,822, entitled “Method and Apparatus for Automated Biopsy and Collection of Soft Tissue,” issued Jun. 18, 1996; U.S. Pat. No. 6,017,316, entitled “Vacuum Control System and Method for Automated Biopsy Device,” issued Jan. 25, 2000; U.S. Pat. No. 6,086,544, entitled “Control Apparatus for an Automated Surgical Biopsy Device,” issued Jul. 11, 2000; U.S. Pat. No. 6,432,065, entitled “Method for Using a Surgical Biopsy System with Remote Control for Selecting an Operational Mode,” issued Aug. 13, 2002; U.S. Pat. No. 7,442,171, entitled “Remote Thumbwheel for a Surgical Biopsy Device,” issued Oct. 8, 2008; U.S. Pat. No. 7,854,706, entitled “Clutch and Valving System for Tetherless Biopsy Device,” issued Dec. 1, 2010; U.S. Pat. No. 7,914,464, entitled “Surgical Biopsy System with Remote Control for Selecting an Operational Mode,” issued Mar. 29, 2011; U.S. Pat. No. 7,938,786, entitled “Vacuum Timing Algorithm for Biopsy Device,” issued May 10, 2011; U.S. Pat. No. 8,083,687, entitled “Tissue Biopsy Device with Rotatably Linked Thumbwheel and Tissue Sample Holder,” issued Dec. 21, 2011; U.S. Pat. No. 8,118,755, entitled “Biopsy Sample Storage,” issued Feb. 21, 2012; U.S. Pat. No. 8,206,316, entitled “Tetherless Biopsy Device with Reusable Portion,” issued Jun. 26, 2012; U.S. Pat. No. 8,491,496, entitled “Biopsy Device with Sample Storage,” issued on Jul. 23, 2013; U.S. Pat. No. 8,702,623, entitled “Biopsy Device with Discrete Tissue Chambers,” issued Apr. 22, 2014; U.S. Pat. No. 8,764,680, entitled “Handheld Biopsy Device with Needle Firing,” issued Jul. 1, 2014; U.S. Pat. No. 9,095,326, entitled “Biopsy System with Vacuum Control Module,” issued Aug. 4, 2015; U.S. Pat. No. 9,326,755, entitled “Biopsy Device Tissue Sample Holder with Bulk Chamber and Pathology Chamber,” issued May 3, 2016; U.S. Pat. No. 9,345,457, entitled “Presentation of Biopsy Sample by Biopsy Device,” issued May 24, 2016; U.S. Pat. No. 10,905,404, entitled “Tissue Sample Holder with Enhanced Features,” issued on Feb. 2, 2021; and U.S. Pat. No. 11,504,101, entitled “Biopsy Device with Remote Multi-Chamber Tissue Sample Holder” issued on Nov. 22, 2022. The disclosure of each of the above-cited U.S. Patents is incorporated by reference herein.

Additional exemplary biopsy devices and biopsy system components are disclosed in U.S. Pat. Pub. No. 2006/0074345, entitled “Biopsy Apparatus and Method,” published Apr. 6, 2006, now abandoned; U.S. Pat. Pub. No. 2010/0152610, entitled “Hand Actuated Tetherless Biopsy Device with Pistol Grip,” published Jun. 17, 2010; U.S. Pat. Pub. No. 2010/0160819, entitled “Biopsy Device with Central Thumbwheel,” published Jun. 24, 2010, now abandoned; U.S. Pat. Pub. No. 2012/0283563, entitled “Biopsy Device with Manifold Alignment Feature and Tissue Sensor,” published Nov. 8, 2012, now abandoned; U.S. Pat. App. No. 2013/0150751, entitled “Biopsy Device With Slide-In Probe,” published Jun. 13, 2013; and U.S. Pat. App. No. 2013/0324882, entitled “Control for Biopsy Device,” published Dec. 5, 2013. The disclosure of each of the above-cited U.S. Patent Application Publications, U.S. Non-Provisional Patent Applications, and U.S. Provisional Patent Applications is incorporated by reference herein.

While several systems and methods have been made and used for obtaining a biopsy sample, it is believed that no one prior to the inventor has made or used the invention described in the appended claims.

### BRIEF DESCRIPTION OF THE DRAWINGS

While the specification concludes with claims which particularly point out and distinctly claim this technology, it is believed this technology will be better understood from the following description of certain examples taken in conjunction with the accompanying drawings, in which like reference numerals identify the same elements and in which:

FIG. 1A depicts a perspective view of an exemplary biopsy system;

FIG. 1B depicts a detailed perspective view of a needle of a biopsy device of the biopsy system of FIG. 1A;

FIG. 2 depicts a perspective view of an exemplary tissue handling assembly that may be readily incorporated into the system of FIG. 1;

FIG. 3 depicts a perspective view of a tissue sample holder of the tissue handling assembly of FIG. 2;

FIG. 4 depicts a cross-sectional view of a sample tray of the tissue sample holder of FIG. 3, the cross-section taken along line 4-4 of FIG. 3;

FIG. 5 depicts a front elevational view of the tissue sample holder of FIG. 2;

FIG. 6 depicts a detailed perspective view of a tube articulator of the tissue sample holder of FIG. 2; and

FIG. 7 depicts a perspective view of the tissue sample holder of FIG. 2 with the tube articulator of FIG. 6 being moved for tissue collection;

FIG. 8 depicts another perspective view of the tissue sample holder of FIG. 2, with the tube articulator of FIG. 6 remaining stationary while other components of the tissue handling assembly move for tissue collection.

The drawings are not intended to be limiting in any way, and it is contemplated that various embodiments of the technology may be carried out in a variety of other ways, including those not necessarily depicted in the drawings. The accompanying drawings incorporated in and forming a part of the specification illustrate several aspects of the present technology, and together with the description serve to explain the principles of the technology; it being understood, however, that this technology is not limited to the precise arrangements shown.

### DETAILED DESCRIPTION

The following description of certain examples of the technology should not be used to limit its scope. Other examples, features, aspects, embodiments, and advantages of the technology will become apparent to those skilled in the art from the following description, which is by way of illustration, one of the best modes contemplated for carrying out the technology. As will be realized, the technology described herein is capable of other different and obvious aspects, all without departing from the technology. Accordingly, the drawings and descriptions should be regarded as illustrative in nature and not restrictive.

### I. Overview of Exemplary Biopsy System

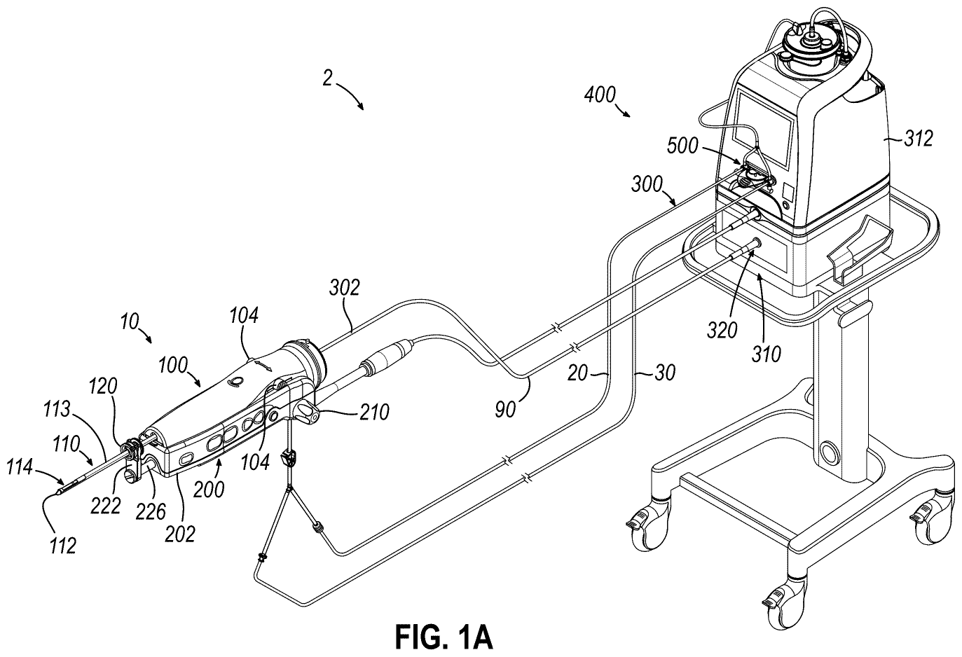

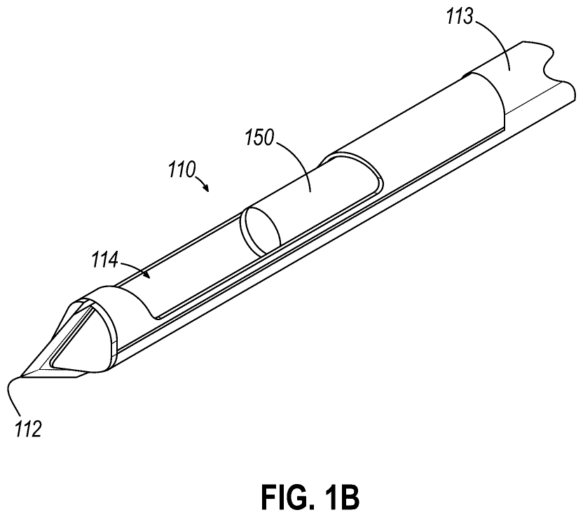

FIG. 1A depicts an exemplary biopsy system (2) including a biopsy device (10) and a vacuum control module (400). Biopsy device (10) of the present example includes a probe (100) and a holster (200). A needle (110) extends distally from probe (100), and is inserted into a patient's tissue to obtain tissue samples. These tissue samples are communicated through needle and into a tissue handling assembly (300) with a tissue transport tube (302) connected at the proximal end of probe (100), as will also be described in greater detail below. It should also be understood that the use of the term “holster” herein should not be read as requiring any portion of probe (100) to be inserted into any portion of holster (200). For instance, in the present example, holster (200) includes a set of prongs (not shown) that are received by at least a portion of probe (100) to releasably secure probe (100) to holster (200). Probe (100) optionally may include one or more resilient tabs (104) that may be pressed inwardly to disengage the prongs, such that a user may simultaneously depress both tabs (104) then pull probe (100) rearwardly and away from holster (200) to decouple probe (100) from holster (200). Of course, a variety of other types of structures, components, features, etc. (e.g., bayonet mounts, latches, clamps, clips, snap fittings, etc.) may be used to provide removable coupling of probe (100) and holster (200). Furthermore, in some biopsy devices (10), probe (100) and holster (200) may be of unitary or integral construction, such that the two components cannot be separated. By way of example only, in versions where probe (100) and holster (200) are provided as separable components, probe (100) may be provided as a disposable component, while holster (200) may be provided as a reusable component. Still other suitable structural and functional relationships between probe (100) and holster (200) will be apparent to those of ordinary skill in the art in view of the teachings herein.

Biopsy device (10) of the present example is configured to mount to a table or fixture, and be used under stereotactic guidance. Of course, biopsy device (10) may instead be used under ultrasound guidance, MRI guidance, PEM guidance, BSGI guidance, or otherwise. It should also be understood that biopsy device (10) may be sized and configured such that biopsy device (10) may be operated by a single hand of a user. In particular, a user may grasp biopsy device (10), insert needle (110) into a patient's breast, and collect one or a plurality of tissue samples from within the patient's breast, all with just using a single hand. Alternatively, a user may grasp biopsy device (10) with more than one hand and/or with any desired assistance. In some settings, the user may capture a plurality of tissue samples with just a single insertion of needle (110) into the patient's breast. Such tissue samples may be pneumatically deposited within at least a portion of tissue handling assembly (300), and later retrieved from tissue handling assembly (300) for further analysis. While examples described herein often refer to the acquisition of biopsy samples from a patient's breast, it should be understood that biopsy device (10) may be used in a variety of other procedures for a variety of other purposes and in a variety of other parts of a patient's anatomy (e.g., prostate, thyroid, etc.). Various exemplary components, features, configurations, and operabilities of biopsy device (10) will be described in greater detail below; while other suitable components, features, configurations, and operabilities will be apparent to those of ordinary skill in the art in view of the teachings herein.

Holster (200) of the present example includes an outer housing (202) that encloses various components that are used to drive various components of probe (100) for the collection of tissue samples. Although not shown, it should be understood that holster (200) of the present example includes one or more gears (not shown) that mesh with corresponding gears of probe (100). In particular, these gears are exposed through an upper portion of outer housing (202) to mesh with corresponding gears of probe (100) when probe (100) and holster (200) are coupled together. This configuration permits holster (200) to communicate rotary motion to probe (100), thereby driving various components of probe (100) for the collection of tissue samples. For instance, the gears may drive an actuation assembly associated with a hollow tubular cutter (150) (see FIG. 1B) within needle (110) to sever tissue samples received within a lateral aperture (114) defined by needle (110). Likewise, other gears may be employed to rotate needle (110).

As noted above, in some examples a gear associated with holster (200) can provide rotation of needle (110) relative to probe (100). In the present example, this rotation is manually initiated by rotating knob (210). In particular, knob (210) is coupled with the gear associated with rotation of needle (110) by a series of gears (not shown) and shafts (not shown), such that rotation of knob (210) rotates needle (110). By way of example only, such a needle rotation mechanism may be constructed in accordance with the teachings of U.S. Pub. No. 2008/0214955, the disclosure of which is incorporated by reference herein. As another merely illustrative example, a needle rotation mechanism may be constructed in accordance with the teachings of U.S. Pub. No. 2010/0160819, the disclosure of which is incorporated by reference herein. In some other versions, needle (110) is rotated by a motor. In still other versions, needle (110) is simply rotated by rotating thumbwheel (116). Various other suitable ways in which rotation of needle (110) may be provided will be apparent to those of ordinary skill in the art in view of the teachings herein. It should also be understood that some versions may provide no rotation of needle (110).

Holster (200) also includes a firing rod (226) and fork (222), which couple with needle (110) and fire needle (110) distally. By way of example only, such firing may be useful in instances where biopsy device (10) is mounted to a stereotactic table fixture or other fixture, with tip (112) adjacent to a patient's breast, such that the needle firing mechanism may be activated to drive needle (110) into the patient's breast. The needle firing mechanism may be configured to drive needle (110) along any suitable range of motion, to drive tip (112) to any suitable distance relative to fixed components of probe (100).

In the present example, the needle firing mechanism is coupled with needle (110) via a firing rod (226) and a firing fork (222). Firing rod (226) and firing fork (222) are unitarily secured together. Firing fork (222) includes a pair of prongs that receive hub member (120) of needle (110) therebetween. The prongs of firing fork (222) are positioned between an annular flange and a thumbwheel of hub member (120), such that needle (110) will translate unitarily with firing rod (226) and fork (222). The prongs nevertheless removably receive hub member (120), such that fork (222) may be readily secured to hub member (120) when probe (100) is coupled with holster (200); and such that hub member (120) may be readily removed from fork (222) when probe (100) is decoupled from holster (200). The prongs are also configured to permit hub member (120) to rotate between the prongs. Other suitable components, configurations, and relationships will be apparent to those of ordinary skill in the art in view of the teachings herein. The internal components of the needle firing mechanism of the present example are configured and arranged as described in U.S. Pat. No. 8,858,465, entitled “Biopsy Device with Motorized Needle Firing,” issued on Oct. 14, 2014, the disclosure of which is incorporated by reference herein.

Holster (200) of the present example is powered by one or more motors (not shown) contained within outer housing (202). These motors are generally configured to drive one or more gears to thereby rotate and translate a tubular cutter (150) disposed within needle (110). Holster (200) also includes a motor (not shown) that is operable to drive firing rod (226), to thereby arm and fire needle (110). All motors referred to herein are contained within holster (200) in the present example and receive power from vacuum control module (400) via cable (90). In addition, data may be communicated between vacuum control module (400) and holster (200) via cable (90). In some other versions, one or more motors are powered by one or more batteries located within holster (200) and/or probe (100). It should therefore be understood that, as with other components described herein, cable (90) is merely optional. As yet another merely illustrative variation, motors may be powered pneumatically, such that cable (90) may be substituted with a conduit communicating a pressurized fluid medium to holster (200). As still other merely illustrative variation, cable (90) may include one or more rotary drive cables that are driven by motors that are located external to holster (200). It should also be understood that two or three of the motors may be combined as a single motor. Other suitable ways in which various the motors may be driven will be apparent to those of ordinary skill in the art in view of the teachings herein.

Probe (100) of the present example includes a needle (110) extending distally from probe (100) that is inserted into a patient's tissue to obtain tissue samples. Such tissue samples are transported proximally through needle (110) and into transport tube (302), where the tissue samples may be deposited within at least a portion of tissue handling assembly (300), as will be described in greater detail below. Vacuum control module (400) is coupled with probe (100) via a valve assembly (500) and tubes (20, 30), which is operable to selectively provide vacuum, saline, atmospheric air, and venting to probe (100). Although the present example is shown with a pair of tubes (20, 30), it should be understood that in some examples one tube may be omitted in lieu of a tissue transport tube (302) discussed below. The internal components of the valve assembly of the present example are configured and arranged as described in U.S. Pub. No. 2013/0218047, entitled “Biopsy Device Valve Assembly,” published Aug. 22, 2013, the disclosure of which is incorporated by reference herein.

As described above, probe (100) may include one or more gears to mesh with corresponding gears of holster (200). These gears are operable to drive a cutter actuation mechanism in probe (100). Probe (100) may also include another gear that is configured to mesh with a corresponding gear of holster (200) to thereby rotate needle (110).

Needle (110) of the present example comprises a cannula (113) having a tissue piercing tip (112), a lateral aperture (114) located proximal to tip (112), and a hub member (120). Tissue piercing tip (112) is configured to pierce and penetrate tissue, without requiring a high amount of force, and without requiring an opening to be pre-formed in the tissue prior to insertion of tip (112). Alternatively, tip (112) may be blunt (e.g., rounded, flat, etc.) if desired. By way of example only, tip (112) may be configured in accordance with any of the teachings in U.S. Pat. No. 8,801,742, entitled “Needle Assembly and Blade Assembly for Biopsy Device,” issued on Aug. 12, 2014, the disclosure of which is incorporated by reference herein. As another merely illustrative example, tip (112) may be configured in accordance with at least some of the teachings in U.S. Pat. No. 9,486,186, entitled “Biopsy Device with Slide-In Probe,” issued on Nov. 8, 2016, the disclosure of which is incorporated by reference herein. Other suitable configurations that may be used for tip (112) will be apparent to those of ordinary skill in the art in view of the teachings herein.

Lateral aperture (114) is sized to receive prolapsed tissue during operation of device (10). Although not shown, it should be understood that a hollow tubular cutter (150) having a sharp distal edge is located within needle (110). Cutter (150) is operable to rotate and translate relative to needle (110) and past lateral aperture (114) to sever a tissue sample from tissue protruding through lateral aperture (114). For instance, cutter (150) may be moved from an extended position to a retracted position, thereby “opening” lateral aperture (114) to allow tissue to protrude therethrough; then from the retracted position back to the extended position to sever the protruding tissue. As will be described in greater detail below, needle (110) may be rotated to orient lateral aperture (114) at any desired angular position about the longitudinal axis of needle (110). Such rotation of needle (110) is facilitated in the present example by hub member (120), which is described in greater detail below.

In some examples, needle (110) also includes a longitudinal wall (not shown) extending proximally from the proximal portion of tip (112). In such examples, the wall may only extend along a length less than the full length of cannula (113). However, in other examples such a wall may extend the full length of cannula (113) if desired. Where needle (110) includes the wall, the wall may define a two-lumen configuration within needle (110). In examples where the wall only extends for a portion of needle (110), it should be understood that at least a portion of cutter (150) may also define the two-lumen configuration of needle (110). Furthermore, to facilitate fluid flow between lumens, the wall may include a plurality of openings (not shown). An example of such a configuration is disclosed in U.S. Pat. No. 7,918,803, entitled “Methods and Devices for Automated Biopsy and Collection of Soft Tissue,” issued Apr. 5, 2011, the disclosure of which is incorporated by reference herein. Of course, as with any other component described herein, any other suitable configurations may be used.

Hub member (120) of the present example is overmolded about needle (110), such that hub member (120) and needle (110) rotate and translate unitarily with each other. By way of example only, needle (110) may be formed of metal, and hub member (120) may be formed of a plastic material that is overmolded about needle (110) to unitarily secure and form hub member (120) to needle (110). Hub member (120) and needle (110) may alternatively be formed of any other suitable material(s), and may be secured together in any other suitable fashion. In the present example, hub member (120) defines a thumbwheel feature to provide manual rotation of needle. Various other suitable ways in which manual rotation of needle (110) may be provided will be apparent to those of ordinary skill in the art in view of the teachings herein. It should also be understood that rotation of needle (110) may be automated in various ways, including but not limited to the various forms of automatic needle rotation described in various references that are cited herein.

As noted above, needle (110) contains a hollow tubular cutter that is operable to simultaneously translate and rotate relative to needle (110) to sever a tissue sample from tissue protruding through lateral aperture (114). Although not shown, it should be understood that in some examples such a cutter (150) may be operatively coupled to a cutter drive mechanism disposed within probe. Such a cutter drive mechanism may be in communication with one or more gears, which may mesh with one or more corresponding gears of holster (200). The cutter drive mechanism may thus be driven by gears of holster (200) to simultaneously rotate and translate cutter (150) disposed within needle (110). In some examples, cutter (150) drive mechanism may include various threaded and keyed features to facilitate simultaneous rotation and translation of cutter (150). In such examples, a single rotatory input may be converted by the cutter drive mechanism into both rotation and translation of cutter (150). Alternatively, in other examples, rotation and translation of cutter (150) may be supplied by separate rotary inputs. In yet other examples, both rotation and translation of cutter (150) may be provided by two rotary inputs acting together such as by two gears moving at different rotational speeds. In some versions, the foregoing cutter actuation components are further configured in accordance with at least some of the teachings of U.S. Pat. No. 9,345,457, entitled “Presentation of Biopsy Sample by Biopsy Device,” issued on May 24, 2016, the disclosure of which is incorporated by reference herein. As yet another merely illustrative example, cutter (150) disposed within needle (110) may be rotated and/or translated using pneumatic motors, etc. Still other suitable ways in which cutter (150) disposed within needle (110) may be actuated will be apparent to those of ordinary skill in the art in view of the teachings herein.

### II. Exemplary Tissue Handling Assembly

Tissue handling assembly (300) is best seen in FIGS. 1A-4. As seen in FIG. 1A, at least a portion of tissue handling assembly (300) is incorporated into vacuum control module (400). However, it should be understood that in other examples, tissue handling assembly (300) may be entirely independent and separate from vacuum control module (400). Tissue handling assembly (300) of the present example includes a tissue transport tube (302), and a tissue handler (310). Tissue transport tube (302) extends from tissue handler (310) to biopsy device (10). As will be described in greater detail below, tissue transport tube (302) is generally configured to receive tissue samples from cutter (150) disposed within needle (110) and to communicate such tissue samples from biopsy device (10) to tissue handler (310).

Tissue handler (310) generally includes an outer housing (312) and a collection drawer (320). As will be described in greater detail below, tissue handler (310) is generally configured to receive a plurality of tissue samples from tissue transport tube (302). The tissue samples are then generally arranged in a predetermined configuration. As will also be described in greater detail below, tissue hander (310) may include various sample analysis features to provide procedure room analysis of tissue samples collected within tissue handler (310). Suitable analysis features may include, among other things, x-ray transmitters and receivers, CCD cameras for visual inspection, bioimpedance sensors, and/or etc.

Collection drawer (320) is received within a portion of outer housing (312). Collection drawer (320) is generally translatable into and out of outer housing (312) to provide access to the interior of collection drawer (320) and thereby permit removal of tissue samples and/or various components from tissue handler (310), as will be described in greater detail below. In some examples, collection drawer (320) is manually operable to translate relative to outer housing (312). In other examples, translation of collection drawer (310) is powered by motor driven or pneumatically driven assemblies to provide automatic or semi-automatic translation of collection drawer (310).

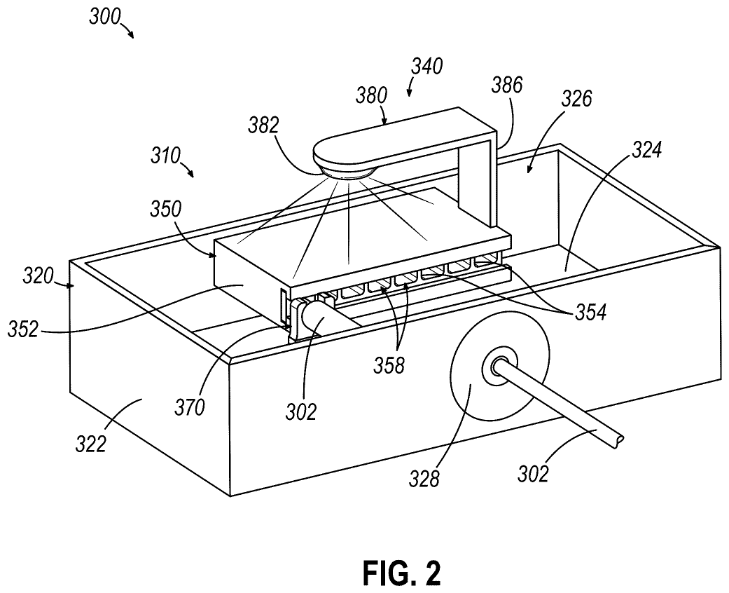

As best seen in FIG. 2, the interior of collection drawer (320) includes a plurality of sidewalls (322) and a floor (324) defining an interior space (326). Tissue transport tube (302) is configured to communicate with the interior space (326) of collection drawer (320) through a tube port (328) disposed in at least one sidewall (322) of collection drawer (320). Although not shown, it should be understood that in some examples, sidewalls (322) and/or floor (324) may include one or more ports to provide draining of fluid from collection drawer (320). For instance, in some instances biopsy device (10) is used with saline or other fluid mediums. Through the tissue sample collection process, at least some refuse fluids from the biopsy procedure may flow into interior space (326) of collection drawer (320). Thus, it may be desirable in such examples to include drainage ports through sidewalls (322) and/or floor (324).

In the present example, collection drawer (320) contains a tissue sample holder (340) within interior space (326) defined by sidewalls (322) and floor (324). Tissue sample holder (340) of the present example is generally in communication with tissue transport tube (302) to receive a plurality of tissue samples in a predetermined arrangement or configuration. Tissue sample holder (340) of the present example incudes a sample tray (350) and an analysis assembly (380) integrated into at least a portion of sample tray (350). Sample tray (350) and analysis assembly (380) together define an elongate rectangular structure configured to receive tissue samples in a linear arrangement relative to a longitudinal axis defined by sample tray (350). As will be described in greater detail below, such collected samples may then be imaged or otherwise analyzed via analysis assembly (380). As will also be described in greater detail below, sample tray (350) is generally of a cassette-like configuration such that sample tray (350) or other components associated with sample tray (350) may move within collection drawer (320) to successively collect tissue samples within discrete portions of sample tray (350). In some examples, tissue sample holder (340) may further include one or more tray containers, covers, or casings, which may be configured to removably receive sample tray (350). In such examples, such tray containers may be desirable to control vacuum applied to sample tray (350) or to manage fluid communicated into one or more portions of sample tray (350). Of course, in other versions, such tray containers are entirely optional and may be omitted.

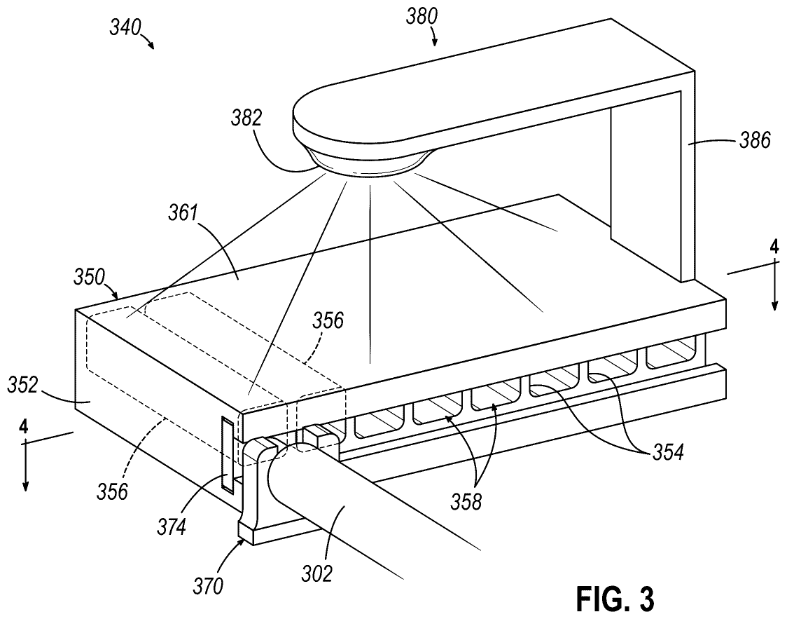

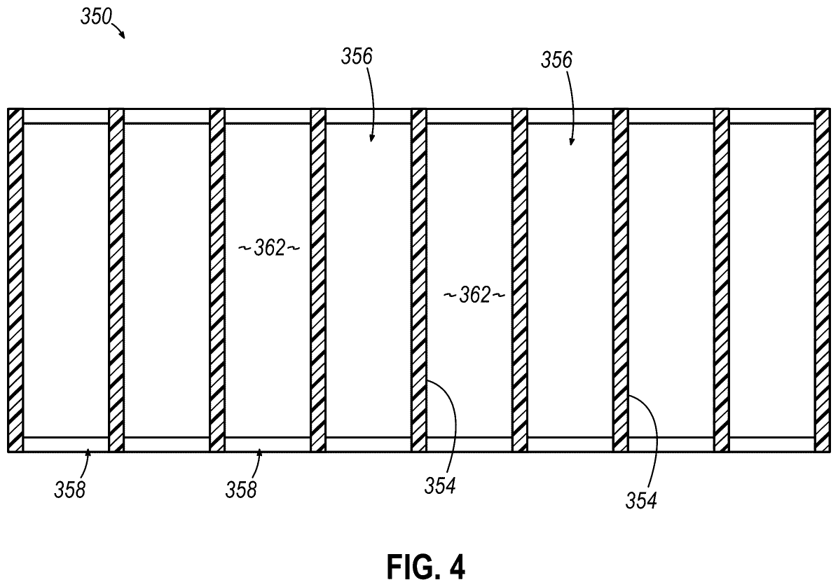

As best seen in FIGS. 3 and 4, sample tray (350) of the present example includes a rectangular body (352) with a plurality of divider walls (354) defining a plurality of sample chambers (356). A front portion of rectangular body (352) defines a plurality of receiving openings (358) in communication with each respective sample chamber (356). Optionally, a rear portion of rectangular body (352) defines a plurality of vacuum openings (not shown), also in communication with each respective sample chamber (356). Each receiving opening (358) is configured to independently communicate with tissue transport tube (302) so that one or more tissue samples may be communicated into a respective sample chamber (356). Correspondingly, in examples including vacuum openings, each vacuum opening is configured to communicate with a vacuum tube (not shown) or other source of vacuum disposed opposite of tissue transport tube (302) such that vacuum may be communicated into a respective sample chamber (356) to draw one or more tissue samples into the respective sample chamber (356) under vacuum.

An upper portion of rectangular body (352) is generally closed or otherwise covered by a top surface (361). In other examples, the upper portion of rectangular body (352) is open or otherwise exposed to the exterior of sample tray (350) such that each sample chamber (356) may be accessible from the upper portion of rectangular body (352). In yet other examples, one or more portions of rectangular body (352) may be configured to be selectively covered such as by a door, cover, casing, and/or etc. Such selective covering may be desirable in some examples to control the flow of vacuum through sample chambers (356), particularly during transport of one or more tissue samples. Thus, in in some examples, each sample chamber (356) may be independently coverable so only the particular sample chamber (356) receiving one or more tissue samples may be covered. In still other examples, such covering may be omitted and vacuum may be controlled by other components such as fixtures or housings associated with collection drawer (320).

A lower portion of rectangular body (352) includes a floor (362) (see FIG. 4). In the present example, floor (362) is formed of a generally solid material. While floor (362) of the present example is generally non-permeable, floor (362) may have at least some permeability in some examples. For instance, in some examples floor (362) includes a semi-permeable membrane or a mesh configured to permit fluids to pass through floor (362). Such permeability may be desirable to control fluid entering sample tray (350). Thus, in other examples, floor (362) is generally non-permeable, but may include other fluid management features such as ports, valves, and/or etc. In still other examples, fluid may be controlled through vacuum tube (304).

As best seen in FIG. 3, sample tray (350) is disposed at a proximal end of tissue transport tube (302). Transport tube (302) and is generally aligned a sampling axis extending generally perpendicular relative to the longitudinal axis of sample tray (350). In some examples, a vacuum tube (not shown) may likewise be aligned with the sampling axis. Thus, in such examples, tissue transport tube (302) and the vacuum tube are each configured to simultaneously be in communication with a selected sample chamber (356) of the plurality of sample chambers (356) depending on the axial position of sample tray (350) relative to tissue transport tube (302).

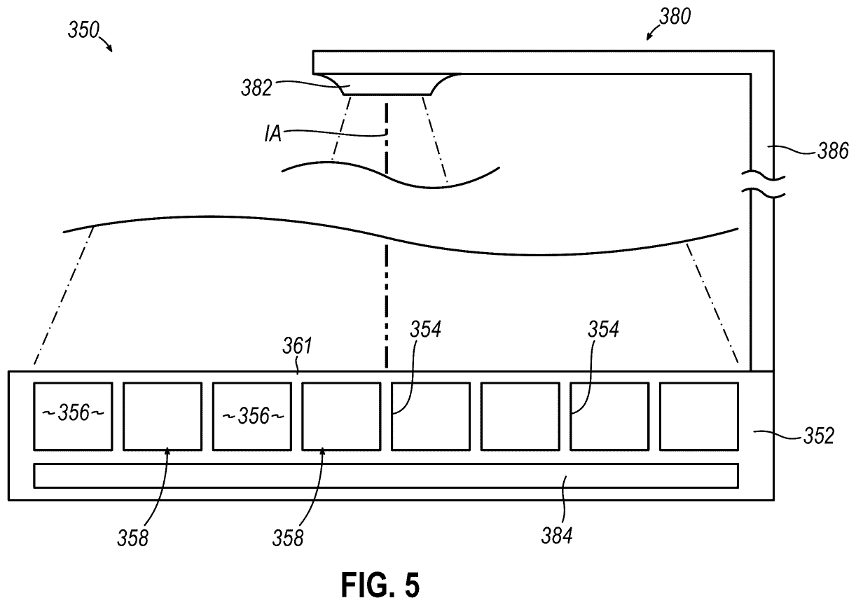

As best seen in FIG. 5, tissue sample holder (340) includes integration of analysis assembly (380) into a portion of sample tray (350). In particular, analysis assembly (380) includes one or more imaging elements configured to image or otherwise analyze tissue samples contained within sample tray (350). In the present example, analysis assembly (380) is configured for x-ray imaging. Thus, analysis assembly (380) includes a source (382) and a detector (384) oriented on opposing sides of sample tray (350). In the present example, detector (384) is integrated into a portion of floor (362). Meanwhile, source (382) is oriented above top surface (361) using a source arm (386). Source arm (386) extends upwardly and laterally relative to top surface (361). The upward extension component of source arm (386) is configured to position source (382) a fixed distance from sample tray (350). The lateral extension component of source arm (386) is configured to position source (382) at about the center point of sample tray (350). Although source arm (386) is generally an integral component with respect to sample tray (350), it should be understood that in other examples, source arm (386) may be a separate component fixedly secured to sample tray (350) or any other component having a generally fixed spatial relationship relative to sample tray (350).

Source (382) and detector (384) are together configured to image each sample chamber (356) of sample tray (350) using a single image or analysis acquisition. In particular, detector (384) spans underneath each sample chamber (356) to overlap with each sample chamber (356). Meanwhile, source (382) is positioned a distance from sample tray (350) to project a triangular beam with a width corresponding to the width of detector (384). Source (382) and detector (384) are together centered along an imaging axis (IA) corresponding the center of sample tray (350).

In the present example, source (382) and detector (384) are both configured for x-ray imaging. As will be described in greater detail below, such x-ray imaging may include real-time or digital x-ray imaging. Such imaging modalities may be desirable to simultaneously image all sample chambers (356), while using software or image processing to visualize each sample chamber (356) individually. Of course, in other examples, various alternative imaging or other analysis modalities may be used. It should be understood that in examples using different analysis modalities, different source (382) or detector (384) configurations may be used. In such examples, either source (382) or detector (384) can be omitted. In other examples, source (382) and detector (384) may be combined into a single component oriented on either side of sample tray (350).

As noted above, each sample chamber (356) is configured to selectively communicate with tissue transport tube (302) so one or more tissue samples may be received within each sample chamber (356). To facilitate such selectable communication, tissue sample holder (340) of the present example includes a tube articulator (370) (also referred to as a tube slider) proximate receiving openings (358). Tube articulator (370) is generally configured to join tissue transport tube (302) with sample tray (350) while permitting movement of transport tube (302) relative to sample tray (350).

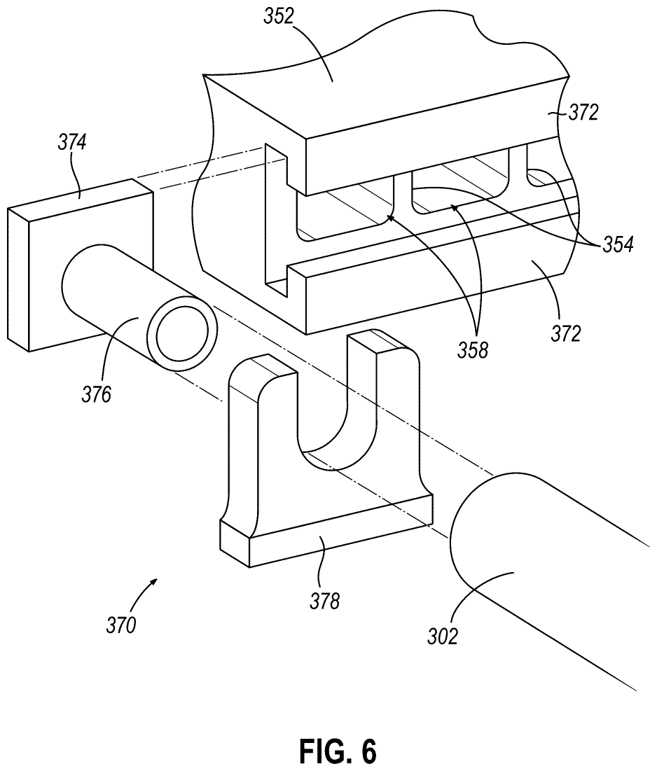

As best seen in FIG. 6, tube articulator (370) includes a rail (372), a sliding seal (374), and a tube driver (378). Rail (372) extends along the length of sample tray (350) above and below receiving openings (358) with receiving openings (358) being position between each of an upper portion and lower portion of rail (372). Rail (372) is configured to slidably receive a portion of seal (374) such that seal (374) may slide along the length of rail (372) and/or the longitudinal axis of sample tray (350).

Seal (374) is generally configured to sealingly engage the distal surface of sample tray (350) while also being movable relative to sample tray (350). Seal (374) defines a generally rectangular shape corresponding to the shape and/or configuration of rail (372). Seal (374) further includes a hollow tube protrusion (376) extending from a distal surface of seal (374), which is configured for receipt within tissue transport tube (302) to thereby couple tissue transport tube (302) to seal (374).

Tube driver (378) defines a generally fork-shaped configuration, which is configured to receive tissue transport tube (302) between each tine of the fork-shape of tube driver (378). Tube driver (378) is configured to exert a force on tissue transport tube (302) and seal (374) (via tube protrusion (376)) to drive tissue transport tube (302) and seal (374) along rail (372). Although not shown, it should be understood that tube driver (378) is configured to couple to other drive features such as linear actuators, rack-based actuators, cables, and/or etc. to facilitate movement of tissue transport tube (302) relative to sample tray (350). Such mechanisms may additionally be motor driven or mechanically driven (e.g., by spring-loaded mechanisms). In yet other examples, translation of tube actuator (370) may be fixed relative to other components such as collection drawer (320), while sample tray (350) itself may be moved by such mechanisms. In yet other examples, any movement may be controlled manually by an operator.

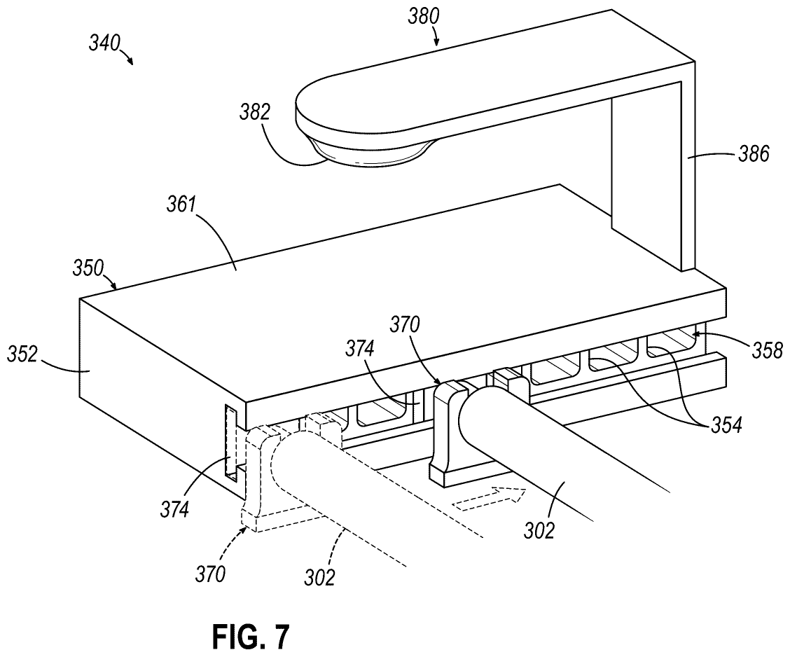

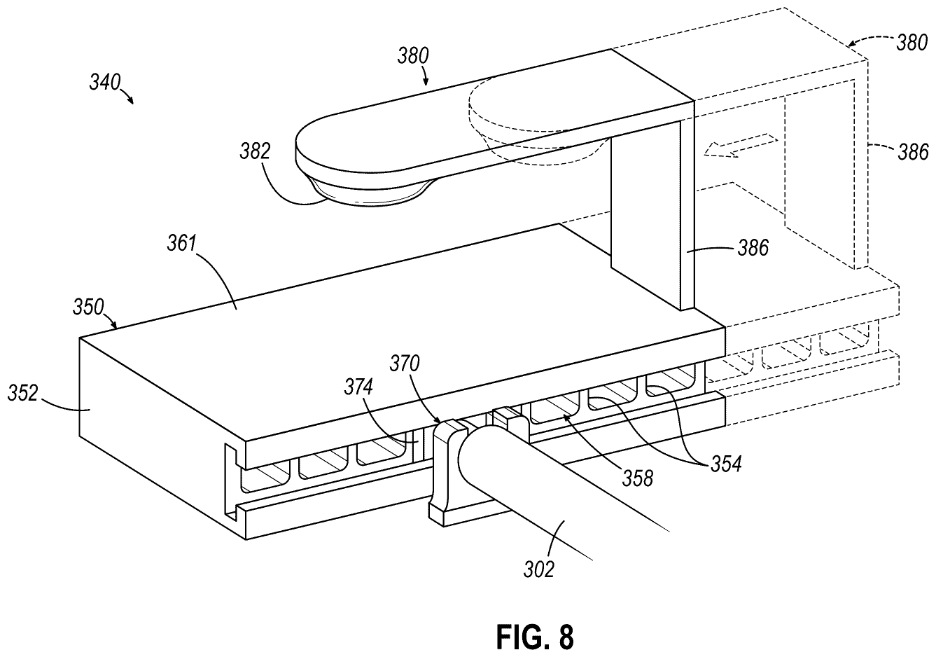

As best seen in FIGS. 7 and 8, in use, sample tray (350) is configured to receive tissue samples in each sample chamber (356) and image each received tissue sample upon receipt. In some examples, tissue transport tube (302) may move relative to sample tray (350) via tube articulator (370) as shown in FIG. 7. Meanwhile, in other examples, tissue transport tube (302) may remain fixed, while sample tray (350) moves relative to tissue transport tube (302) as shown in FIG. 8.

In use, sample tray (350) may be initially aligned relative to tissue transport tube (302) so that a predetermined initial sample chamber (356) is aligned with tissue transport tube (302). In the present example, the predetermined initial sample chamber (356) corresponds to the left-most sample chamber (356), although any other sample chamber (356) may be used in other examples.

Regardless of the particular predetermined initial sample chamber (356), a tissue sample may be communicated into the predetermined initial sample chamber (356) via tissue transport tube (302) to transport the tissue sample from biopsy device (10) and into the predetermined initial sample chamber (356) using vacuum and/or saline. Although not shown, it should be understood sample tray (350) can include one or more tissue stopping strctures configured to prevent the tissue sample from progressing out of a given sample chamber (356), thereby holding the tissue sample within the given sample chamber (356).

Once the tissue sample is received within the predetermined initial sample chamber (356), sample tray (350) or tissue transport tube (302) may be advanced to position another sample chamber (356) into alignment with tissue transport tube (302). In the present example, this advancement is show as progressing tissue transport tube (302) or sample tray (350) to the right or left, respectively. In some examples, this progression may be sequential such that the next adjacent sample chamber (356) is aligned with tissue transport tube (302). In other examples, the progression may be any other suitable sequence (e.g., moving from one end to the other).

Once sample tray (350) is advanced, another tissue sample may be received in the next aligned sample chamber (356). This process may be repeated until all sample chambers (356) are filled. Alternatively, this process may continue until a desired number of tissue samples have been collected.

At any stage during the collection of tissue samples described above, imaging or other analysis may be performed. As described above, movement of sample tray (350) is fixed or coordinated with movement of analysis assembly (380). Thus, analysis assembly (380) is positioned to image or otherwise analyze sample tray (350) at any point during sample acquisition. As also described above, such imaging or other analysis generally corresponds to the whole of sample tray (350). Thus, any collected tissue samples are imaged or otherwise analyzed simultaneously. Thus, in some examples, it may be desirable to perform imaging or other analysis after all desired samples have been collected within sample tray (350). In other examples, imaging or analysis may be performed after each sample acquisition or after a predetermined group of sample acquisitions. In still other examples, imaging or analysis may be performed continuously regardless of sample acquisition.

Regardless of when imaging or other analysis occurs, such imaging or other analysis may include at least some image processing steps. For instance, in the present example, analysis assembly (380) is configured for digital or real-time x-ray imaging. Thus, such x-ray imaging includes acquisition of a whole sample tray image. The whole sample tray image may then be manipulated to produce individual sample images of each collected tissue sample. Such individual sample images may then be analyzed by an operator either in real time or at the conclusion of the biopsy procedure. When real time imaging is used, such real time imaging analysis may be used to inform an operator for acquisition of subsequent tissue samples.

### III. Exemplary Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

A biopsy system, the biopsy system comprising: a biopsy device, the biopsy device including: a probe, a needle extending from the probe, and a cutter, the cutter being movable relative to the needle to sever one or more tissue samples; a tissue transport tube configured to couple to a portion of the biopsy device to communicate the one or more severed tissue samples from the biopsy device; and a tissue sample holder separate from the biopsy device and configured to couple to the tissue transport tube to receive the one or more severed tissue samples, the tissue sample holder including a sample tray and an analysis assembly, the sample tray being configured to receive the one or more severed tissue samples, the sample tray having a fixed spatial relationship with respect to the analysis assembly.

### Example 2

The biopsy system of Example 1, the sample tray including a rectangular body defining a plurality of sample chambers arranged along a longitudinal axis of the rectangular body.

### Example 3

The biopsy system of Example 2, the analysis assembly being positioned relative to the sample tray to analyze each sample chamber of the plurality of sample chambers simultaneously.

### Example 4

The biopsy system of Examples 2 or 3, the sample tray defining a plurality of receiving openings, each receiving opening corresponding to a sample chamber of the plurality of sample chambers, each receiving opening being configured to selectively communicate with the tissue transport tube.

### Example 5

The biopsy system of Example 4, the tissue sample holder further including a tube actuator, the tube actuator being configured to drive relative movement between the tissue transport tube and the sample tray.

### Example 6

The biopsy system of Example 5, the tube actuator including a seal, the seal being configured to slide relative to the sample tray between each receiving opening of the plurality of sample openings.

### Example 7

The biopsy system of claim 5, the tube actuator including a seal, the seal being configured to slide along a rail defined by a portion of the sample tray to translate relative to the sample tray between each receiving opening of the plurality of sample openings.

### Example 8

The biopsy system of any of Examples 1 through 7, the analysis assembly including an x-ray source and an x-ray detector.

### Example 9

The biopsy system of Example 8, the x-ray source and the x-ray detector having a fixed spatial relationship with respect to the sample tray.

### Example 10

The biopsy system of Examples 8 or 9, the x-ray source and the x-ray detector both being integral with a portion of the sample tray.

### Example 11

The biopsy system of Example 1, the sample tray including a rectangular body defining a plurality of sample chambers arranged along a longitudinal axis of the rectangular body, the analysis assembly including an x-ray source and an x-ray detector, the x-ray detector defining a length, the length of the x-ray detector corresponding to a length defined by the plurality of sample chambers.

### Example 12

The biopsy system of Example 1, the sample tray including a rectangular body defining a plurality of sample chambers arranged along a longitudinal axis of the rectangular body, the analysis assembly including an x-ray source and an x-ray detector, the x-ray detector at least a portion of the x-ray detector being positioned below each sample chamber of the plurality of sample chambers.

### Example 13

The biopsy system of Examples 1 through 12, further comprising a control module, the tissue sample holder being disposed within a portion of the control module.

### Example 14

The biopsy system of any of Examples 1 through 13, the sample tray being configured to move relative to the tissue transport tube.

### Example 15

The biopsy system of any of Examples 1 through 13, the tissue transport tube being configured to move relative to the sample tray.

### Example 16

An apparatus for use with a biopsy device, the apparatus comprising: a tissue transport tube, the tissue transport tube being configured to receive and transport one or more tissue samples acquired by the biopsy device; a tissue sample holder, the tissue sample holder including a sample tray, the sample tray including a plurality of sample chambers arranged linearly, each sample chamber being in selective communication with the tissue transport tube to receive the one or more tissue samples acquired by the biopsy device from the tissue transport tube; and an analysis assembly including one or more imaging elements, the imaging elements being positioned relative to the sample tray to image each sample chamber of the plurality of sample chambers simultaneously.

### Example 17

The apparatus of Example 16, the analysis assembly further including an arm, the arm being fixedly secured to the sample tray and configured to position at an imaging element of the one or more imaging elements in a fixed position relative to the sample tray.

### Example 18

The apparatus of Examples 16 or 17, the sample tray and the analysis assembly being together configured to move relative to the tissue transport tube.

### Example 19

The apparatus of any of Examples 16 through 18, the one or more imaging elements including an x-ray source and an x-ray detector.

### Example 20

A method of analyzing a tissue sample, the method comprising: transporting a first tissue sample acquired by a biopsy device through a tissue transport tube into a first sample chamber of a sample tray; moving the sample tray relative to the tissue transport tube to align a second sample chamber with the tissue transport tube; transporting a second tissue sample acquired by the biopsy device through the tissue transport tube into the second sample chamber of the sample tray; imaging the first tissue sample and the second tissue sample simultaneously to form a whole sample tray image; and acquiring an individual image of each of the first tissue sample and the second tissue sample from the whole sample tray image.

### Example 21

The method of Example 20, the step of moving the sample tray relative to the tissue transport tube further including translating the tissue tray along a longitudinal axis define by the tissue tray.

### Example 22

The method of Examples 20 or 21, the step of imaging the first tissue sample and the second tissue sample including imaging via x-ray imaging.

### Example 23

The method of Examples 20 or 21, the step of imaging the first tissue sample and the second tissue sample including imaging via digital x-ray imaging.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Embodiments of the present invention have application in conventional endoscopic and open surgical instrumentation as well as application in robotic-assisted surgery.

By way of example only, embodiments described herein may be processed before surgery. First, a new or used instrument may be obtained and if necessary cleaned. The instrument may then be sterilized. In one sterilization technique, the instrument is placed in a closed and sealed container, such as a plastic or TYVEK bag. The container and instrument may then be placed in a field of radiation that can penetrate the container, such as gamma radiation, x-rays, or high-energy electrons. The radiation may kill bacteria on the instrument and in the container. The sterilized instrument may then be stored in the sterile container. The sealed container may keep the instrument sterile until it is opened in a medical facility. A device may also be sterilized using any other technique known in the art, including but not limited to beta or gamma radiation, ethylene oxide, or steam.

Embodiments of the devices disclosed herein can be reconditioned for reuse after at least one use. Reconditioning may include any combination of the steps of disassembly of the device, followed by cleaning or replacement of particular pieces, and subsequent reassembly. In particular, embodiments of the devices disclosed herein may be disassembled, and any number of the particular pieces or parts of the devices may be selectively replaced or removed in any combination. Upon cleaning and/or replacement of particular parts, embodiments of the devices may be reassembled for subsequent use either at a reconditioning facility, or by a surgical team immediately prior to a surgical procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

1. A biopsy system, the biopsy system comprising:
(a) a biopsy device, the biopsy device including:
(i) a probe,
(ii) a needle extending from the probe, and
(iii) a cutter, the cutter being movable relative to the needle to sever one or more tissue samples;
(b) a tissue transport tube configured to couple to a portion of the biopsy device to communicate the one or more severed tissue samples from the biopsy device; and
(c) a tissue sample holder separate from the biopsy device and configured to couple to the tissue transport tube to receive the one or more severed tissue samples, the tissue sample holder including a sample tray and an analysis assembly, the sample tray being configured to receive the one or more severed tissue samples, the sample tray having a fixed spatial relationship with respect to the analysis assembly.

2. The biopsy system of claim 1, the sample tray including a rectangular body defining a plurality of sample chambers arranged along a longitudinal axis of the rectangular body.

3. The biopsy system of claim 2, the analysis assembly being positioned relative to the sample tray to analyze each sample chamber of the plurality of sample chambers simultaneously.

4. The biopsy system of claim 2, the sample tray defining a plurality of receiving openings, each receiving opening corresponding to a sample chamber of the plurality of sample chambers, each receiving opening being configured to selectively communicate with the tissue transport tube.

5. The biopsy system of claim 4, the tissue sample holder further including a tube actuator, the tube actuator being configured to drive relative movement between the tissue transport tube and the sample tray.

6. The biopsy system of claim 5, the tube actuator including a seal, the seal being configured to slide relative to the sample tray between each receiving opening of the plurality of sample openings.

7. The biopsy system of claim 5, the tube actuator including a seal, the seal being configured to slide along a rail defined by a portion of the sample tray to translate relative to the sample tray between each receiving opening of the plurality of sample openings.

8. The biopsy system of claim 1, the analysis assembly including an x-ray source and an x-ray detector.

9. The biopsy system of claim 8, the x-ray source and the x-ray detector having a fixed spatial relationship with respect to the sample tray.

10. The biopsy system of claim 8, the x-ray source and the x-ray detector both being integral with a portion of the sample tray.

11. The biopsy system of claim 1, the sample tray including a rectangular body defining a plurality of sample chambers arranged along a longitudinal axis of the rectangular body, the analysis assembly including an x-ray source and an x-ray detector, the x-ray detector defining a length, the length of the x-ray detector corresponding to a length defined by the plurality of sample chambers.

12. The biopsy system of claim 1, the sample tray including a rectangular body defining a plurality of sample chambers arranged along a longitudinal axis of the rectangular body, the analysis assembly including an x-ray source and an x-ray detector, the x-ray detector at least a portion of the x-ray detector being positioned below each sample chamber of the plurality of sample chambers.

13. The biopsy system of claim 1, further comprising a control module, the tissue sample holder being disposed within a portion of the control module.

14. The biopsy system of claim 1, the sample tray being configured to move relative to the tissue transport tube.

15. The biopsy system of claim 1, the tissue transport tube being configured to move relative to the sample tray.

16. An apparatus for use with a biopsy device, the apparatus comprising:
(a) a tissue transport tube, the tissue transport tube being configured to receive and transport one or more tissue samples acquired by the biopsy device;
(b) a tissue sample holder, the tissue sample holder including a sample tray, the sample tray including a plurality of sample chambers arranged linearly, each sample chamber being in selective communication with the tissue transport tube to receive the one or more tissue samples acquired by the biopsy device from the tissue transport tube; and
(c) an analysis assembly including one or more imaging elements, the imaging elements being positioned relative to the sample tray to image each sample chamber of the plurality of sample chambers simultaneously.

17. The apparatus of claim 16, the analysis assembly further including an arm, the arm being fixedly secured to the sample tray and configured to position at an imaging element of the one or more imaging elements in a fixed position relative to the sample tray.

18. The apparatus of claim 16, the sample tray and the analysis assembly being together configured to move relative to the tissue transport tube.

19. The apparatus of claim 16, the one or more imaging elements including an x-ray source and an x-ray detector.

20. A method of analyzing a tissue sample, the method comprising:
(a) transporting a first tissue sample acquired by a biopsy device through a tissue transport tube into a first sample chamber of a sample tray;
(b) moving the sample tray relative to the tissue transport tube to align a second sample chamber with the tissue transport tube;
(c) transporting a second tissue sample acquired by the biopsy device through the tissue transport tube into the second sample chamber of the sample tray;
(d) imaging the first tissue sample and the second tissue sample simultaneously to form a whole sample tray image; and
(e) acquiring an individual image of each of the first tissue sample and the second tissue sample from the whole sample tray image.

21.-23. (canceled)
